(19) United States
(12) Patent Application Publication
(10) Pub. No.: US 2018/0000393 A1
(43) Pub. Date: Jan. 4, 2018

(54) MULTIPLE PHYSIOLOGICAL SIGNALS SENSING CHIP AND THE MULTIPLE PHYSIOLOGICAL SIGNALS SENSING METHOD THEREOF

(71) Applicant: NATIONAL TSING HUA UNIVERSITY, Hsinchu (TW)

(72) Inventors: Chih-Cheng Hsieh, Hsinchu (TW); Albert Yen-Chih Chiou, Hsinchu (TW)

(21) Appl. No.: 15/263,189

(22) Filed: Sep. 12, 2016

(30) Foreign Application Priority Data
Jul. 4, 2016 (TW) 105121064

(51) Int. Cl. A61B 5/1455; A61B 5/0205; A61B 5/00

(52) U.S. Cl. CPC A61B 5/14552; A61B 5/6826; A61B 5/02055; A61B 2562/0238

(57) ABSTRACT

A multiple physiological signals sensing chip is provided. The multiple physiological signals sensing chip includes a substrate, a first light-emitting diode, a second light-emitting diode, a sensing array, and a processing unit. The substrate includes a contact surface touched by a finger. The first and second light-emitting diodes respectively emit red light and infrared light to the finger. The sensing array senses the red light or the infrared light reflected or refracted from the finger to obtain first physiological sensing signals according to a first sensing period or senses the red light and the infrared light reflected or refracted from the finger to obtain second physiological sensing signals according to a second sensing period. The first sensing period is shorter than the second sensing period. The processing unit respectively processes the first and second physiological sensing signals to obtain spatial information and energy information corresponding to the finger.

### CROSS REFERENCE TO RELATED APPLICATION

The present application is based on, and claims priority from, TW Application Number 105121064, filed on Jul. 4, 2016, the invention of which is hereby incorporated by reference herein in its entirety.

### TECHNICAL FIELD

The invention relates to a sensing device used to sense multiple physiological signals, and more particularly to sense multiple physiological signals using a single chip.

### BACKGROUND

Currently, fingerprint sensing techniques adopted in commercial electronic devices mostly use capacitive sensing methods, and blood oxygen or heartbeat sensing techniques adopted in commercial electronic devices mostly use optical sensing methods. Due to the rapid development of semiconductor technology, development of related electronic products towards the direction of integrating multiple applications. In view of this, the present invention provides a multiple physiological signals sensing chip and a corresponding multiple physiological signals sensing method.

### SUMMARY

An embodiment of the present invention provides a multiple physiological signals sensing chip. The multiple physiological signals sensing chip comprises a substrate, at least one first light-emitting diode, at least one second light-emitting diode, a sensing array, and a processing unit. The substrate comprises a contact surface touched by a finger of a user. The at least one first light-emitting diode is used to emit red light to the finger. The at least one second light-emitting diode is used to emit infrared light to the finger. The sensing array senses the red light or the infrared light reflected or refracted from the finger to obtain a plurality of first physiological sensing signals according to a first sensing period or senses the red light and the infrared light reflected or refracted from the finger to obtain a plurality of second physiological sensing signals according to a second sensing period, wherein the first sensing period is shorter than the second sensing period. The processing unit is connected to the at least one first light-emitting diode, the at least one second light-emitting diode and the sensing array, wherein the processing unit operates the at least one first light-emitting diode, the at least one second light-emitting diode and the sensing array according to the first sensing period and the second sensing period; and wherein the processing unit processes the first physiological sensing signals to obtain spatial information corresponding to the finger and processes the second physiological sensing signals to obtain energy information corresponding to the finger.

An embodiment of the present invention provides a multiple physiological signals sensing method. The multiple physiological signals sensing method comprises touching, by a finger of a user, a contact surface of a substrate; emitting red light to the finger; emitting infrared light to the finger; sensing the red light or the infrared light reflected or refracted from the finger to obtain a plurality of first physiological sensing signals according to a first sensing period; sensing the red light and the infrared light reflected or refracted from the finger to obtain a plurality of second physiological sensing signals according to a second sensing period, wherein the first sensing period is shorter than the second sensing period; processing the first physiological sensing signals to obtain spatial information corresponding to the finger; and processing the second physiological sensing signals to obtain energy information corresponding to the finger.

### BRIEF DESCRIPTION OF THE DRAWINGS

The present invention can be more fully understood by reading the subsequent detailed description and examples with references made to the accompanying drawings, wherein:

FIG. 1A and FIG. 1B are block diagrams showing a multiple physiological signals sensing chip 10 according to a first embodiment of the present invention.

FIG. 2 is a three-dimension (3D) schematic diagram showing a sensing array 140 according to a second embodiment of the present invention.

FIG. 3 is a 3D schematic diagram showing the sensing array 140 according to a third embodiment of the present invention.

FIG. 4 shows a flow diagram of a multiple physiological signals sensing method according to a fourth embodiment of the present invention.

### DETAILED DESCRIPTION

The following description is of the best-contemplated mode of carrying out the present invention. This description is made for the purpose of illustrating the general principles of the present invention and should not be taken in a limiting sense. The scope of the present invention is best determined by reference to the appended claims.

The optical sensing method utilized in the present invention provides a sensing chip integrating multiple physiological signal sensing functions such as a sensing chip integrating a fingerprint sensing function and a heartbeat sensing function.

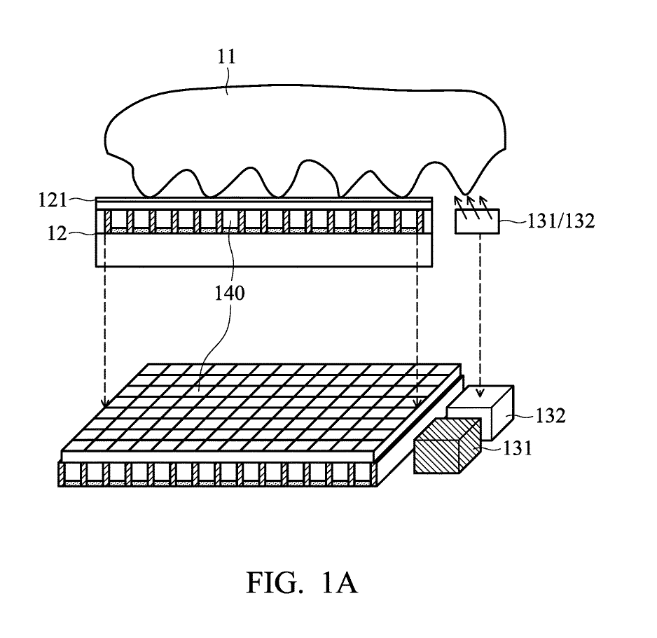

FIG. 1A and FIG. 1B are block diagrams showing a multiple physiological signals sensing chip 10 for sensing multiple physiological signals according to a first embodiment of the present invention. As shown in FIG. 1A and FIG. 1B, in the first embodiment of the present invention, the multiple physiological signals sensing chip 10 comprises a substrate 12, at least one first light-emitting diode 131, at least one second light-emitting diode 132, an image sensor 14, a processing unit 15 and a diode driving circuit 16. The substrate 12 comprises a contact surface 121 which can be touched by a finger 11 of a user. The at least one first light-emitting diode 131 is used to emit red light to the finger 11. The at least one second light-emitting diode 132 is used to emit infrared light to the finger 11. The image sensor 14 comprises a sensing array 140, a readout circuit 142 and a body temperature 143. The readout circuit 142 is respectively connected to the sensing array 140 and the body temperature 143. The sensing array 140 is composed of a plurality of photosensitive diodes 141 (not shown in FIG. 1A and FIG. 1B), and the body temperature 143 is composed of a third light-emitting diode 133 and a corresponding photosensitive diode 144 (not shown in FIG. 1A and FIG. 1B), wherein the third light-emitting diode 133 is used to emit the infrared light having a longer wavelength to the finger 11 and the photosensitive diode 144 is used to sense the infrared light having a longer wavelength reflected or refracted from the finger 11. The processing unit 15 comprises a fingerprint image processing unit 151 and a blood oxygen signal processing unit 152, wherein the fingerprint image processing unit 151 and the blood oxygen signal processing unit 152 are respectively connected to the readout circuit 142 of the image sensor 14. The processing unit 15 is connected to the diode driving circuit 16 and controls the diode driving circuit 16 to drive the first light-emitting diode 131 and the second light-emitting diode 132.

In the first embodiment of the present invention, the photosensitive diodes 141 of the sensing array 140 sense the red light or the infrared light reflected or refracted from the finger 11 according to a first sensing period T_{A}, and the readout circuit 142 of the sensing array 140 reads the corresponding plurality of first physiological sensing signals. The photosensitive diodes 141 of the sensing array 140 also senses the red light or the infrared light reflected or refracted from the finger 11 according to a second sensing period T_{B}, and the readout circuit 142 of the sensing array 140 reads the corresponding plurality of second physiological sensing signals. It should be noted that, in the first embodiment of the present invention, the first sensing period T_{A} is shorter than the second sensing period T_{B}. The processing unit 15 operates the photosensitive diodes 141 and the readout circuit 142 of the sensing array 140 according to the first sensing period T_{A} and the second sensing period T_{B}.

In the first embodiment of the present invention, when the sensing array 140 is operated according to the first sensing period T_{A}, the first sensing period T_{A} is short enough that the energy information of reflected light (or refracted light) sensed by the photosensitive diodes 141 is independent of time. Simultaneously, the processing unit 15 also controls luminous time of the first light-emitting diode 131 according to the first sensing period T_{A}. At this time, the readout circuit 142 of the sensing array 140 reads out the energy information of reflected light (or refracted light) sensed by each of the photosensitive diodes 141.

When the finger 11 touches the contact surface 121 of the substrate 12, fingerprint crests 111 of the finger 11 directly contact the contact surface 121 and fingerprint troughs 112 of the finger 11 do not contact the contact surface 121 due to their containing air. Light from a light source (red light or infrared light) passes through the fingerprint crests 111 to the contact surface 121 of the multiple physiological signals sensing chip 10 so that the photosensitive diodes 141 under the contact surface 121 can sense the light. When light from a light source (red light or infrared light) passes through the fingerprint troughs 112 of the finger 11, the light is scattered by the air contained by the fingerprint troughs 112 in such a way that the photosensitive diodes 141 under the contact surface 121 cannot sense the light.

Because the energy information corresponding to the fingerprint crests 111 and the fingerprint troughs 112 of the finger 11 are different, the energy of the reflected light (or the refracted light) sensed by different positions of the photosensitive diodes 141 of the sensing array 140 are different. Therefore the energy information of the reflected light (or the refracted light) sensed by each of the photosensitive diodes 141 has spatial difference.

Accordingly, in the first embodiment of the present invention, the processing unit 15 adjusts the first sensing period T_{A} so that the first physiological sensing signals of the photosensitive diodes 141 of the sensing array 140 are a plurality of time invariant signals which have a spatial association between each other. For example, the photosensitive diodes 141 of the sensing array 140 sense instantaneously reflected light (or instantaneous refracted light) from the finger 11. At this time, the readout circuit 142 of the sensing array 140 reads out the first physiological sensing signals from the photosensitive diodes 141.

In the first embodiment of the present invention, the fingerprint image processing unit 151 of the processing unit 15 has a gain amplifier, an analog-to-digital circuit and a digital signal processing circuit. Accordingly, the fingerprint image processing unit 151 can process a plurality of sampling signals (i.e. the first physiological sensing signals) read from the readout circuit 142 to obtain spatial information corresponding to the finger 11, wherein the spatial information comprises the positions of the fingerprint crests 111 and the positions of the fingerprint troughs 112. In other words, the spatial information comprises the fingerprint pattern of the finger 11.

In the first embodiment of the present invention, the processing unit 15 adjusts the second sensing period T_{B} so that the second physiological sensing signals sensed by the photosensitive diodes 141 of the sensing array 140 are time varying signals. Simultaneously, the processing unit 15 also controls luminous time of the first light-emitting diode 131 and the second light-emitting diode 132 according to the second sensing period T_{B}.

Since the second physiological sensing signals are required to be the time varying signals but not the time invariant signals, the second sensing period T_{B} needs to be longer than the first sensing period T_{A}. For example, the photosensitive diodes 141 of the sensing array 140 sense the reflected light (or the refracted light) from the finger 11 within a period of time. At this time, the readout circuit 142 of the sensing array 140 determines, according to the second sensing period T_{B}, total energy information of the reflected light (or the refracted light) sensed by the photosensitive diodes 141, wherein the total energy information is a continuous signal which is time varying and uncorrelated to the positions. Therefore the readout circuit 142 of the sensing array 140 can readout the second physiological sensing signals represented by the continuous signal from the photosensitive diodes 141. For example, the red light and the infrared light respectively correspond to two second physiological sensing signals.

In the first embodiment of the present invention, the blood oxygen signal processing unit 152 of the processing unit 15 has a gain amplifier, an analog-to-digital circuit and a digital signal processing circuit. Accordingly, the blood oxygen signal processing unit 152 can process the two second physiological sensing signals read from the readout circuit 142 (i.e. the two second physiological sensing signals corresponding to the red light and the infrared light) to obtain energy information from the finger 11, wherein the energy information comprises blood oxygen information of the finger 11 and heartbeat information of the finger 11.

In the first embodiment of the present invention, the blood oxygen signal processing unit 152 further operates the body temperature sensor 143 of the image sensor 14 according to a third sensing period T_{C}, wherein the third sensing period T_{C} is longer than the first sensing period T_{A}. For example, the body temperature sensor 143 of the image sensor 14 senses the infrared light reflected or refracted from the finger 11 within a period of time, then the readout circuit 142 can read a third physiological sensing signal represented by a continuous signal from the body temperature sensor 143 according to the third sensing period T_{C}. In the first embodiment of the present invention, the third physiological sensing signal is similar to the second physiological sensing signal, and the third physiological sensing signal is a time varying signal containing the total energy information.

In the first embodiment of the present invention, the first light-emitting diode 131, the second light-emitting diode 132, the image sensor 14, the processing unit 15 and the diode driving circuit 16 can be integrated into a silicon chip with a planar manner. Accordingly, the multiple physiological signals sensing chip 10 of the present invention can obtain the fingerprint pattern, the blood oxygen information and heartbeat information corresponding to the finger 11 using an optical sensing method performed by the sensing array of only one chip.

In addition, it should be noted that the multiple physiological signals sensing chip 10 is required to use at least two light sources with different wavelengths (i.e. there is a need to turn on the first light-emitting diode 131 and the light-emitting diode 132) only when the multiple physiological signals sensing chip 10 needs to obtain the blood oxygen information. In other words, in other embodiments of the present invention, the multiple physiological signals sensing chip 10 can obtain the fingerprint pattern, the body temperature information and the heartbeat information corresponding to the finger 11 with only single-wavelength light source (for example, it only turns on the first light-emitting diode 131 or the light-emitting diode 132).

In other embodiments of the present invention, the multiple physiological signals sensing chip 10 is not limited to using the red light source or the infrared light source. For example, the multiple physiological signals sensing chip 10 may also use another light-emitting diode which emits light having another wavelength and thereby obtain physiological information from the finger 11 (for example, fingerprint pattern, blood oxygen information, body temperature information, and heartbeat information).

Since the body temperature sensor 143 is required to sense infrared light which has a longer wavelength, the third light-emitting diode 133 and the photosensitive diode 144 used in the body temperature sensor 143 are mostly implemented by III-V semiconductors. Then a body temperature sensor 143 implemented by III-V semiconductor is integrated into the multiple physiological signals sensing chip 10 through encapsulation.

In addition, it should be noted that, if the body temperature sensor 143 is implemented by silicon, the body temperature sensor 143 also can be implemented in the same chip (or implemented in the same sensing array 140). At this time, the multiple physiological signals sensing chip 10 can obtain the fingerprint pattern, the blood oxygen information, the body temperature information and the heartbeat information corresponding to the finger 11 by an optical sensing method with the sensing array of only one chip.

In another embodiment of the present invention, the processing unit 15 controls the sensing array 140 operating in the first sensing period T_{A}, the readout circuit 142 of the sensing array 140 reads all the first physiological sensing signals. At this time, the fingerprint image processing unit 151 processes all the first physiological sensing signals to obtain spatial information of the finger 11 (for example, the fingerprint pattern of the finger 11), and the blood oxygen signal processing unit 152 of the processing unit 15 processes all the first physiological sensing signals to obtain a first physiological sensing average signal, wherein the first physiological sensing average signal represents an average value sensed by the photosensitive diodes 141 of the sensing array 140 during the first sensing period T_{A}. Since the first sensing period T_{A} is very short (for example, 5 milliseconds), the first physiological sensing average signal is also a time invariant signal. In addition, the magnitude of the first physiological sensing average signal is related to the sensing time of the sensing array 140.

In the same way, the processing unit 15 controls the sensing array 140 and the diode driving circuit 16 at different sensing times such that the blood oxygen signal processing unit 152 respectively obtains the first physiological sensing average signals. For example, the processing unit 15 controls the sensing array 140 operating in the first sensing period T_{A} in every 100 milliseconds such that the blood oxygen signal processing unit 152 obtains 1000 first physiological sensing average signals in 100 seconds. At this time, since each of the first physiological sensing average signals at different sensing time is distinct, the 1000 first physiological sensing average signals can be considered as a time varying signal. That is, 1000 discrete values form single time varying signal. Then the blood oxygen signal processing unit 152 can process the time varying signal (1000 first physiological sensing average signals) corresponding to the red light and the time varying signal (1000 first physiological sensing average signals) corresponding to the infrared light to obtain the blood oxygen information and the heartbeat information corresponding to the finger 11.

Accordingly, in the above embodiment of the present invention, the image sensor 14 of the multiple physiological signals sensing chip 10 operates with an identical sensing period (for example, it always operates with the first sensing period T_{A}) to obtain the fingerprint pattern, the blood oxygen information and the heartbeat information corresponding to the finger 11.

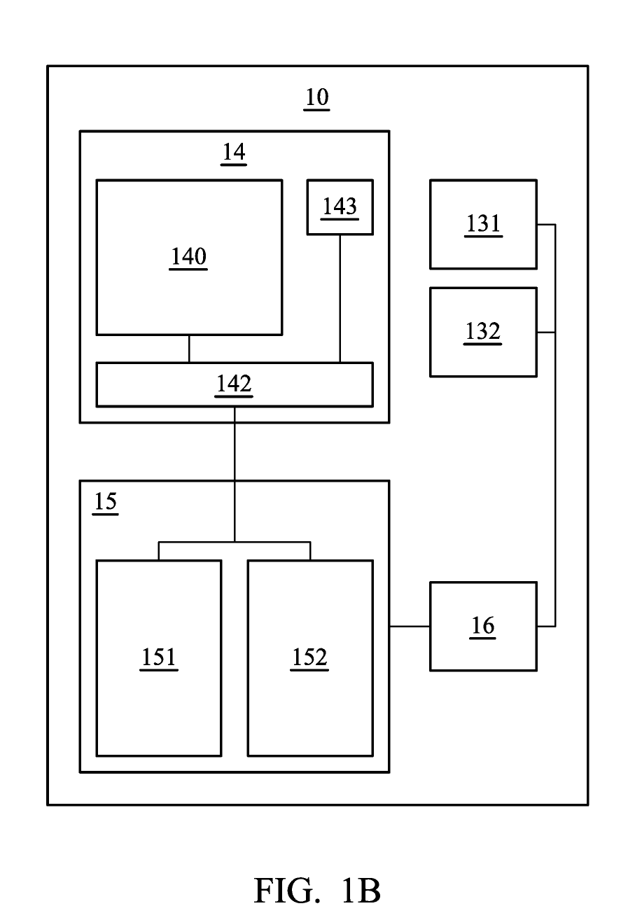

FIG. 2 is a 3D schematic diagram showing a sensing array 140 according to a second embodiment of the present invention. In the second embodiment of the present invention, in order to let the infrared light/red light more uniformly reflect/refract to the sensing array, six first light-emitting diodes 131 and six second light-emitting diodes 132 are uniformly disposed around the sensing array 140.

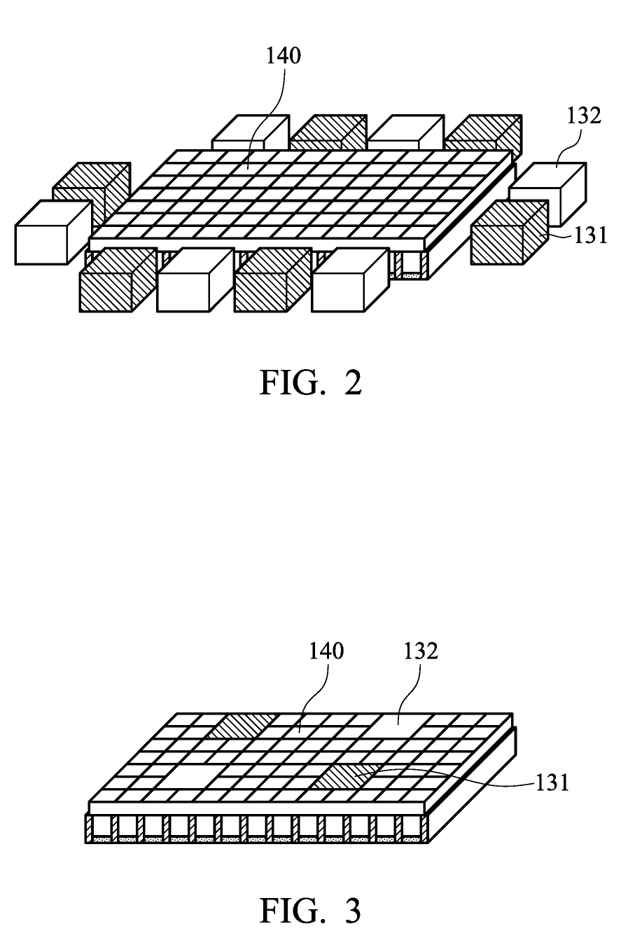

FIG. 3 is a 3D schematic diagram showing the sensing array 140 according to a third embodiment of the present invention. In the third embodiment of the present invention, in order to reduce the designed area of the multiple physiological signals sensing chip 10, the first light-emitting diodes 131 and the second light-emitting diodes 132 are integrated into the sensing array 140. At this time, the sensing array 140 is large enough so that loss of pixels due to the above configuration (integrating it into the sensing array 140) is not enough to influence fingerprint recognition.

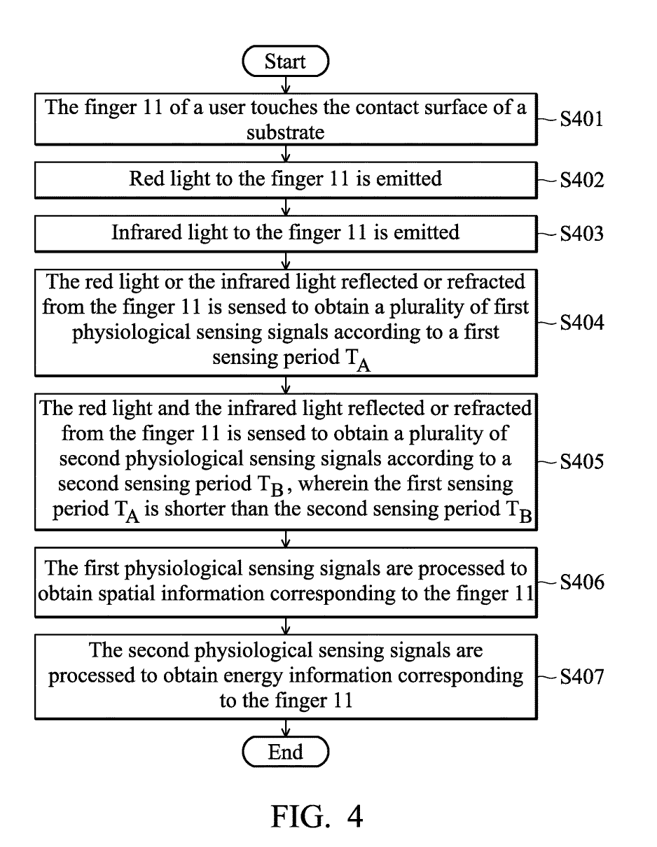

FIG. 4 shows a flow diagram of a multiple physiological signals sensing method according to a fourth embodiment of the present invention. In step S401, the finger 11 of a user touches the contact surface of a substrate. In step S402, red light to the finger 11 is emitted. In step S403, infrared light to the finger 11 is emitted. In step S404, the red light or the infrared light reflected or refracted from the finger 11 is sensed to obtain a plurality of first physiological sensing signals according to a first sensing period T_{A}. In step S405, the red light and the infrared light reflected or refracted from the finger 11 is sensed to obtain a plurality of second physiological sensing signals according to a second sensing period T_{B}, wherein the first sensing period T_{A} is shorter than the second sensing period T_{B}. In step S406, the first physiological sensing signals are processed to obtain spatial information corresponding to the finger 11. In step S407, the second physiological sensing signals are processed to obtain energy information corresponding to the finger 11.

The foregoing outlines features of several embodiments so that those skilled in the art may better understand the aspects of the present invention. Those skilled in the art should appreciate that they may readily use the present invention as a basis for designing or modifying other processes and structures for carrying out the same purposes and/or achieving the same advantages of the embodiments introduced herein. Those skilled in the art should also realize that such equivalent constructions do not depart from the spirit and scope of the present invention, and that they may make various changes, substitutions, and alterations herein without departing from the spirit and scope of the present invention.

What is claimed is:

1. A multiple physiological signals sensing chip, comprising:
a substrate, comprising a contact surface touched by a finger of a user;
at least one first light-emitting diode, used to emit red light to the finger;
at least one second light-emitting diode, used to emit infrared light to the finger;
a sensing array, sensing the red light or the infrared light reflected or refracted from the finger to obtain a plurality of first physiological sensing signals according to a first sensing period or sensing the red light and the infrared light reflected or refracted from the finger to obtain a plurality of second physiological sensing signals according to a second sensing period, wherein the first sensing period is shorter than the second sensing period; and
a processing unit, connected to the at least one first light-emitting diode, the at least one second light-emitting diode and the sensing array, wherein the processing unit operates the at least one first light-emitting diode, the at least one second light-emitting diode and the sensing array according to the first sensing period and the second sensing period; and
wherein the processing unit processes the first physiological sensing signals to obtain spatial information corresponding to the finger and processes the second physiological sensing signals to obtain energy information corresponding to the finger.

2. The multiple physiological signals sensing chip of claim 1, wherein the processing unit adjusts the first sensing period such that the first physiological sensing signals sensed by the sensing array are time invariant signals; and
wherein the processing unit adjusts the second sensing period such that the second physiological sensing signals sensed by the sensing array are time varying signals.

3. The multiple physiological signals sensing chip of claim 2, wherein the processing unit further processes the first physiological sensing signals to obtain a first physiological sensing average signal;
wherein the processing unit operates the sensing array at different time points to obtain the first physiological sensing average signal corresponding to each time point; and
wherein the processing unit processes the first physiological sensing average signals to obtain blood oxygen information of the finger and heartbeat information of the finger.

4. The multiple physiological signals sensing chip of claim 1, wherein the spatial information is a fingerprint pattern of the finger; and
wherein the energy information comprises blood oxygen information of the finger and heartbeat information of the finger.

5. The multiple physiological signals sensing chip of claim 1, further comprising:
a body temperature sensor, connected to the processing unit, wherein the processing unit operates the body temperature sensor according to a third sensing period and the sensing period is longer than the first sensing period; and
wherein the body temperature sensor senses the infrared light reflected from the finger to obtain a plurality of third physiological sensing signals according to the third sensing period such that the processing unit processes the third physiological sensing signals to obtain body temperature information of the finger.

6. The multiple physiological signals sensing chip of claim 1, wherein the at least one first light-emitting diode and the at least one second light-emitting diode surround the sensing array.

7. The multiple physiological signals sensing chip of claim 1, wherein the at least one first light-emitting diode and the at least one second light-emitting diode are disposed in the sensing array.

8. A multiple physiological signals sensing method, comprising:
touching, by a finger of a user, a contact surface of a substrate;
emitting red light to the finger;
emitting infrared light to the finger;
sensing the red light or the infrared light reflected or refracted from the finger to obtain a plurality of first physiological sensing signals according to a first sensing period;
sensing the red light and the infrared light reflected or refracted from the finger to obtain a plurality of second physiological sensing signals according to a second sensing period, wherein the first sensing period is shorter than the second sensing period;
processing the first physiological sensing signals to obtain spatial information corresponding to the finger; and
processing the second physiological sensing signals to obtain energy information corresponding to the finger.

9. The multiple physiological signals sensing method of claim 8, further comprising:
adjusting the first sensing period such that the first physiological sensing signals are time invariant signals; and
adjusting the second sensing period such that the second physiological sensing signals are time varying signals.

10. The multiple physiological signals sensing method of claim 9, further comprising:
processing the first physiological sensing signals to obtain a first physiological sensing average signal;
sensing at different time points to obtain the first physiological sensing average signal corresponding to each time point; and
processing the first physiological sensing average signals to obtain blood oxygen information of the finger and heartbeat information of the finger.

11. The multiple physiological signals sensing method of claim 8, wherein the spatial information is a fingerprint pattern of the finger; and
wherein the energy information comprises blood oxygen information of the finger and heartbeat information of the finger.

12. The multiple physiological signals sensing method of claim 8, further comprising:
sensing the infrared light reflected from the finger to obtain a plurality of third physiological sensing signals according to the third sensing period, wherein the sensing period is longer than the first sensing period; and
processing the third physiological sensing signals to obtain body temperature information of the finger.
